(19) United States
(12) Patent Application Publication
(10) Pub. No.: US 2025/0108146 A1
(43) Pub. Date: Apr. 3, 2025

(54) FUNCTIONALIZED AND/OR DOPED FIBERS

(71) Applicant: OKINAWA INSTITUTE OF SCIENCE AND TECHNOLOGY SCHOOL CORPORATION, Okinawa (JP)

(72) Inventors: Lokesh AGRAWAL, Okinawa (JP); Marco TERENZIO, Okinawa (JP)

(21) Appl. No.: 18/729,787

(22) PCT Filed: Jan. 18, 2023
(86) PCT No.: PCT/JP2023/001383

§ 371 Date: Jul. 17, 2024

(30) Foreign Application Priority Data
Jan. 18, 2022 (JP) 2022-005711

(51) Int. Cl. A61L 27/18; A61L 27/54; D01D 5/00; D01F 1/10; D01F 6/62; D04H 1/435; D04H 1/728

(52) U.S. Cl. CPC A61L 27/18; A61L 27/54; D01D 5/0038; D01D 5/0069; D01D 5/0076; D01F 1/10; D01F 6/62; D04H 1/435; D04H 1/728; A61L 2300/204; A61L 2300/412; A61L 2430/32; D10B 2331/04; D10B 2401/063; D10B 2401/12; D10B 2509/00

(57) ABSTRACT

The disclosure provides a fiber, comprising a biocompatible polymer and melanin, or a fibrous assembly comprising the fiber, a system for fabricating a fibrous assembly, a method for fabricating a fibrous assembly, a method of producing a fiber, and a semiconductor.

### FIELD OF INVENTION

The present disclosure relates to, for example, a fiber, fibrous assembly, scaffold, scaffold material, implantable material or implant. The present disclosure further relates to, for example, a method of transplanting a graft material comprising the fiber. The present disclosure further relates to, for example, a system for fabricating a fibrous assembly, a method of fabricating a fibrous assembly or producing a fiber.

### BACKGROUND

Neural tissue engineering (NTE) aims to improve regeneration and restoration of normal neural functions by employing a combination of cells, scaffold and biological cues. Electrical stimuli act as a critical physiological signal and help in regulation of proliferation and function of nerve cells (Prabhakaran et al., 2011; Shi et al., 2008). In addition to be electrically conducting, a successful NTE scaffold also needs to mimic the fibrous nature of the native extracellular matrix (ECM) (Y. Chen et al., 2020). Several technologies are available for the fabrication of nanoscale topography, like polymer de-mixing, phase separation, colloidal lithography, chemical etching and self-assembly (Dalby et al., 2002; Qian & Shen, 2005; Wood, 2007). Electrospinning is relatively simple and inexpensive, and has been successfully employed for the generation of nano-/micro-fibrous scaffolds, which mimic fibrous nature of ECM. These fibrous scaffolds are generally non-conducting and are thus, not suitable to propagate electrical stimulus to nerve cells seeded onto these surfaces (Khorshidi et al., 2016; Vimal et al., 2016). On the other hand, electrically conducting polymers (ECP) are not easy to electrospin. PHB is a natural polymer, which has been used to fabricate the implants to promote the guided growth of axons (Young et al., 2002). Melanin is also a bodily pigment, which has the ability to donate or accept electrons and to interact with free radicals and other reactive species due to the presence of unpaired electrons, thus being conductive in nature (Agrawal et al., 2022; Bettinger et al., 2009; Mostert et al., 2012). Melanin can, thus, act additionally as an antioxidant and po-tentially minimize toxin-induced tissue destruction and inflammation. Therefore, we propose the use melanin for the synthesis of nanofiber scaffolds. Interestingly, melanin presence is not limited to the skin; a pool of melanin, known as neuromelanin, is present inside the brain, mainly in the substantia nigra or the locus coeruleus (Gollion et al., 2020). Studies have shown that neuromelanin concentration increases with age, suggesting a role in neuroprotection (neuromelanin can chelate metals and xenobiotics) or senescence (Haining & Achat-Mendes, 2017).

### BRIEF EXPLANATION OF THE INVENTION

The inventors tested the potential of a new scaffold as a therapeutic approach in regenerative medicine by investigating its behaviors in vitro and in vivo in a mouse model of Spinal Cord Injury (SCI). Pending the success of the aforementioned preliminary investigation phase in animal model, the inventors further explored the effec-tiveness of new scaffolds in patients in collaboration with medical hospitals. In addition, based on the experience in scaffold fabrication with electrospinning, pho-tolithography and 3D printing, the inventors proposed a new electrospinning setup, which allows efficient and reliable 3D printing. This design will provide a new electrospinning setup with upgraded features, like computer aided 3D design, to overcome the limitations of conventional fabrication methods.

The present disclosure provides, for example, a fiber, fibrous assembly, scaffold, scaffold material, implantable material or implant. The present disclosure further provides, for example, a method of transplanting a graft material comprising the fiber. The present disclosure further provides, for example, a system for fabricating a fibrous assembly, a method of fabricating a fibrous assembly or producing a fiber.

The inventors fabricated a novel biomaterial, which can be used for the preparation of implants/conduits for neural tissue engineering. Taking advantage of electrospinning, inventors developed biodegradable and electrically conductive composite polymeric nanofibrous scaffolds, by blending melanin and Poly (3-hydroxybutyrate) (PHB) together with 1.5 mM of 5-hydroxytryptamine (5-HT) at a respective 2:3 ratios. The surface morphology, physio-chemical properties, and conductivity of the resulting fibrous scaffolds were characterized. Our results show that 5-HT/melanin/PHB composite fibers have rough surface with ˜290 nm (mean) diameter. Further, these fibers show higher thermal and mechanical stability with a melting temperature of 179.05° C., a Young's modulus ranging from 10 to 90 MPa, a hydrophilic index of 61.8 +3.4° and a conductivity of 1.3×10³ Scm⁻¹. To prove biocompatibility with neural tissues, human motor neurons and mouse sensory neurons were successfully cultured on the composite fibers. In addition, DRG culture on aligned fibers promoted the vectorized growth of axons along the fibers. These results collectively suggest that 5-HT/melanin/PHB composite fibrous scaffolds are a biodegradable conducting and biocompatible material suitable for neural tissue engineering application. In addition, inventors also propose a new grid base design of electrospinning setup, which allows to print computer aided designs of 3D scaffold and hydrogels to generate implants with controlled 3D shape and well-defined parameters, precisely tailored to fit into the site of injury. It is considered that this setup will be highly advantageous for the development of cost effective and personalized regenerative treatment of various tissues.

### BRIEF DESCRIPTION OF DRAWINGS

FIG. 1 (A) Typical electrospinning set up for the fabrication of fiber. (Ai) Cylindrical target for the collection of random fibers. (Aii) Blade drum target for the collection of aligned fibers. SEM images of random and aligned fibers with ultrastructure of (B) PVA scaffold, (C) Melanin and PHB blend fibers, (D) 5-HT-melanin and PHB blend fibers, and (E) PHB fibers. (F) Bar graph shows diameter of fibers. Data are shown as mean+s.c.m.; asterisks indicate statistical significance (One-way ANOVA with Tukey's post hoc test; **** p<0.0001).

FIG. 2 FTIR UV-Vis spectra of (A) PHB scaffold, (B) Melanin+PHB scaffold, (C) 5-HT-melanin and PHB scaffold, and (D) PVA scaffold with characteristic peaks of the functional groups. XPS spectra for the elemental analysis for (E) CIS, (F) O1S, and (G) NIS. (Gi) shows the atomic percentage of CIS, O1S and NIS in the scaffolds.

FIG. 3 Thermal and mechanical characterization of scaffolds. Differential scanning calorimetry (DSC) curves of a (A) blank sample, (B) 5-HT-melanin and PHB blend fibers, (C) Melanin and PHB blend fibers, (D) PHB fibers and (E) PVA fibers. Heating cycle is represented in red and cooling cycle in blue color. Tm shows the peak of melting temperature. Representative AFM images of (F) 5-HT-melanin and PHB composite fibers, (G) melanin and PHB blended fibers, (H) PHB fibers and (I) PVA fibers showing the height sensor readings and anisotropic distribution of the calculated Young's modulus (DMT modulus). Height sensor scale bar: 5 μm, color-coded scale bar shows height of the scaffold (left panel) and DMT modulus (right panel).

FIG. 4 Conductivity and hydrophilicity measurement. (A) Resistivity and conductivity measurement with 4-probe of 5-HT-melanin and PHB composite fibers lies in semiconductor range. (B-Bi) Contact angle measurement for the wettability/hydrophilicity analysis of PHB scaffold, Melanin+PHB scaffold, 5-HT-melanin and PHB scaffold, and PVA scaffolds. Data are shown as mean+s.c.m.; asterisks indicate statistical significance (One-way ANOVA with Tukey's post hoc test; **** p<0.0001).

FIG. 5 Mouse sensory and hMNs culture. (A-Aii) Confocal images of DRG neurons culture on (A) glass surface, (Ai) random and (Aii) aligned 5-HT-melanin-PHB scaffolding surfaces. (B-Bi) hMNs growing on the (B) 2D control glass surface and (Bi) 5-HT-melanin and PHB blend fibrous surface. Neurons were stained with anti-BIII-tubulin antibody (in red), (A-Bi) scale bar: 50 μm.

FIG. 6A (Hybrid grid based fabrication system) Detailed machine design and in-corporated technology of the hybrid fabrication system. [FIG. 6B] (Hybrid grid based fabrication system) Our proposed fabrication setup also be configured in conventional ES setup with rotating cylinder target can also. [FIG. 6C] (Hybrid grid based fabrication system) Samples of the grid pattern can be used as a collector for the fabrication of different 3D scaffold.

[FIG. 6D] (Hybrid grid based fabrication system) Typical structure of nanogrid (2 cm×2 cm) pattern, fabricated on glass surface by deposition of 4 nm Titanium and 20 nm Gold layers.

FIG. 7 FTIR UV-Vis spectra of (A) PHB(B) Melanin (C) 5-HT, powders mixed with KBr showing characteristic peaks of the functional groups. (D-E) SEM-EDX tracing of 9% PHB fibers (D and Di) confirming the presence of C (73.5%) and O (26%). 5-HT-Melanin-PHB fibers (E and Ei) are characterized by the presence of C (73.3%), O (25.1%) and N (0.7%). Presence of N only in blend fibers confirms the successful blending of 5-HT and melanin in the fibers and documents its distribution. Scale bar: 25 μm.

FIG. 8 Swelling and degradation analysis of PHB and 5-HT-melanin-PHB scaffolds. Increase in weight due to swelling of the fibers of (A) PHB scaffold and (Ai) 5-HT-melanin-PHB scaffold in PBS after 12 h. (B) Comparison of the percentage (%) swelling capacity or degree of swelling of PHB and melanin-PHB scaffold after 12 h. mᵢ=initial weight of the dry scaffold, m, =weight after swelling, and mx=constant residual weight after drying. (A-Ai) AFM images of dry and swelled PHB fibers and 5-HT-melanin-PHB blend fibers after 12 h. Scale bar: 2 μm, color-coded scale bar for height, scale bar: 8 μm. (C-E) Degradation of the mass of (C) PHB scaffold and (G) 5-HT-melanin-PHB scaffold in PBS after 10 days (D10). (H) Comparison of the percentage (%) of degradation of PHB and 5-HT-melanin-PHB scaffold at D10. Data are shown as mean±s.c.m.; asterisks indicate statistical significance (n=12 scaffolds for each condition, Student t-test; ** p<=0.01 and **** p<=0.0001).

### DERAILED EXPLANATION OF THE INVENTION

The term “fiber” as used herein refers to a material having a thread-like structure. The term “fibrous assembly” as used herein refers to a material formed of a plurality of fibers. The term “nano fiber” as used herein refers to a fiber having a sub-micrometer or less than 1000 nm in diameter.

The term “biocompatible” as used herein refers to having no harmful effect on animals such as mammals, preferably humans.

The present disclosure provides a fiber or fibrous assembly. In an embodiment, the fiber can be a biocompatible fiber, a nano fiber, or a biocompatible nano fiber. In a preferable embodiment, the fiber or the biocompatible fiber may be a fiber of a biodegradable polymer, more preferably be a fiber of a natural biodegradable polymer. In a preferable embodiment, the fiber has a conductivity. The conductivity is preferably sufficient for neurons to attach to and/or proliferate on the fiber. The fibrous assembly contains a plurality of fibers, comprising one or more fibers as mentioned above. In a preferable embodiment, the fibrous assembly consists of the fibers as mentioned above.

In a preferable embodiment, examples of the biocompatible polymers include, for example, but not limited to, polyhydroxy alkane (PHA), preferably polyhydroxy butyrate (PHB), more preferably poly (3-hydroxy butyrate).

In a preferable embodiment, the fiber can further comprise melanin. In a preferable embodiment, the fiber can further comprise serotonin (5-HT). In a more preferable embodiment, the fiber can further comprise serotonin and melanin. In the embodiments, the biodegradability of the fiber may preferably increase. In these embodiments, the fiber is made of a polymer, preferably a biocompatible polymer, and melanin and/or 5-HT. In an embodiment, the biocompatible polymer may be a non-conducting polymer, because melanin and 5-TH can impart a conductivity to the fiber in a dose dependent manner.

In a preferable embodiment, the fiber satisfies at least one, two, three, four, five, or all of:
(i) having 100 nm to 2 μm (e.g., 250 nm to 1.5 μm) in diameter;
(ii) having a crystallization temperature of 160° C. to 190° C.;
(iii) having a Young's modulus of 10 MPa to 90 MPa;
(iv) exhibiting a contact angle of less than 90° (e.g., 55° to) 80°;
(v) having a conductivity of 1×10² S/cm to 1×10⁵ S/cm, 1×10² S/cm to 1×10⁴ S/cm, or 1×10² S/cm to 1×10³ S/cm; and
(vi) exhibiting a high biodegradability compared to a fiber that contains no melanin and no serotonin.

In a preferable embodiment, the fiber has 100 nm to 10 μm, 100 nm to 8 μm, 100 nm to 5 μm, 100 nm to 4 μm, 100 nm to 3 μm, or 100 nm to 2 μm (e.g., 250 nm to 1.5 μm, 250 nm to 1 μm, or 500 nm to 1.5 μm) in diameter.

In a preferable embodiment, the fiber has a crystallization temperature of 120° C. to 200° C., 120° C. to 150° C., 160° C. to 190° C., or 140° C. to 180° C.

In a preferable embodiment, the fiber has a Young's modulus of 10 MPa to 90 MPa, 10 MPa to 50 MPa, 40 MPa to 90 MPa, 20 MPa to 80 MPa, or 30 MPa to 80 MPa.

In a preferable embodiment, the fiber has a conductivity sufficient for neurons to proliferate on the fiber. Melanin and 5-HT can impart such a conductivity to a non-conducting fiber such as polyhydroxy alkane (PHA) in a dose dependent manner. Thus, the fiber may preferably comprise a sufficient amount of melanin and/or 5-HT. In a preferable embodiment, the fiber has a conductivity of 1×10² S/cm to 1×10⁵ S/cm, 1×10² S/cm to 1×10+S/cm, or 1×10² S/cm to 1×10³ S/cm.

In a preferable embodiment, the fiber comprises melanin and has a conductivity of 1×10² S/cm to 1×10⁵ S/cm, 1×10² S/cm to 1×10⁴ S/cm, or 1×10² S/cm to 1×10 3 S/cm. In a preferable embodiment, the fiber comprises melanin and 5-HT, and has a conductivity of 1×10² S/cm to 1×10⁵ S/cm, 1×10² S/cm to 1×10+S/cm, or 1×10 2 S/cm to 1×10³ S/cm.

In a preferable embodiment, the fiber has a hydrophilic surface. In an embodiment, the fiber has a hydrophilic surface exhibiting 90 degree or less, 80 degree or less, 70 degree or less, 60 degree or less, 50 degree or less, 40 degree or less, 30 degree or less, 20 degree or less, or 10 degree or less. In an embodiment, the fiber has a hydrophilic surface exhibiting 10 degree or more, 20 degree or more, 30 degree or more, 40 degree or more, 50 degree or more, 60 degree or more, 70 degree or more, or 80 degree or more. In an embodiment, the fiber has a hydrophilic surface exhibiting 10 degree to 90 degree, 10 degree to 50 degree, 50 degree to 90 degree, or 55 degree to 80 degree, 20 degree to 50 degree.

In a preferable embodiment, the fiber is coated with an extracellular matrix, such as collagen, fibronectin, gelatin, and laminin. An extracellular matrix is known to support attachment of animal cells to a surface coated with the extracellular matrix. In a preferable embodiment, the fiber is coated with poly L-lysine. In a preferable cm-bodiment, the fiber is coated with laminin and poly L-lysine. The coating will allow animal cells such as neurons to proliferate on the fiber or fibrous assembly. In these embodiments, the fiber or fibrous assembly comprising the fiber may be suitable for culturing a cell on its surface.

In an embodiment, the present disclosure provides a scaffold or scaffold material, implantable material or implant, comprising a fiber or fibrous assembly of the present disclosure. In an embodiment, the present disclosure provides a non-woven fabric or web, comprising a fiber or fibrous assembly of the present disclosure. The term “non-woven fabric” or “non-woven web” means an article or sheet that has a structure of individual fibers, which are interlaid, for example, in a reticular manner, but not in an identifiable manner. The scaffold or scaffold material can be used for culturing an organ or a tissue in vitro or in vivo. The scaffold or scaffold material can be suitable and/or used for culturing a neuron or a nerve. The scaffold or scaffold material can be suitable and/or used for inducing nerve regeneration in vivo, or in vitro to obtain a graft material.

The present disclosure provides a method of transplanting a graft material in a subject (e.g., human subject) in need thereof. The method may comprise transplanting the graft material to the subject. In an embodiment, the graft material comprises the fiber or fibrous assembly, scaffold or scaffold material, implantable material or implant, comprising a plurality of the fibers of the present disclosure; wherein a plurality of the fibers may form a non-woven fabric or be aligned in a reticular manner in the graft material; and wherein the graft material may further comprise a nerve cell for nerve regeneration on a surface of the graft, thereby optionally inducing nerve regeneration in the subject. In an embodiment, the fiber may comprise melanin and 5-HT, and be coated with an extracellular matrix, preferably laminin, and poly L-lysine. In a preferable embodiment, the fiber is made of PHB supplemented with melanin and 5-HT to have a conductivity.

In an embodiment, the fiber or the assembly may be dried, for example, by natural drying or preferably lyophilization. Thus, the present disclosure provides a dried form of the fiber or assembly, preferably, a lyophilized form of the fiber or assembly. In an embodiment, the fiber or the assembly is suitable for attachment of a cell, for example, a neural stem cell; a neuron such as a sensory neuron, an interneuron, and a motor neuron; a ganglion neuron such as a dorsal root ganglia neuron and trigeminal ganglia neuron; and a glia cell such as an astrocyte, an oligodendrocyte, a microglia, an ependiomocyte, a Schwann cell, and satellite cell. In a preferable embodiment, the cell to be attached may be a motor neuron and a ganglia neuron, more preferably a dorsal root ganglia neuron.

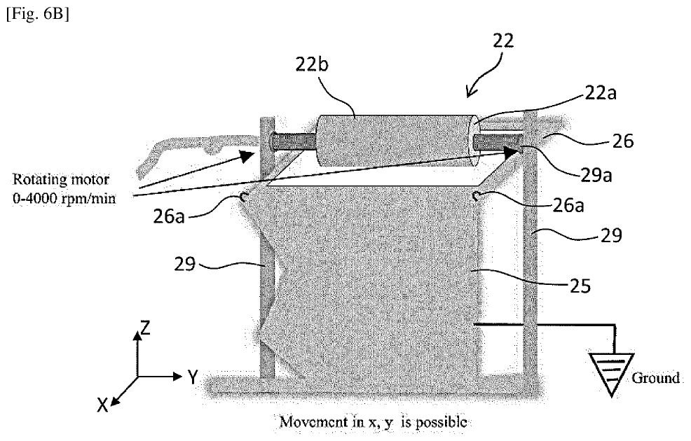

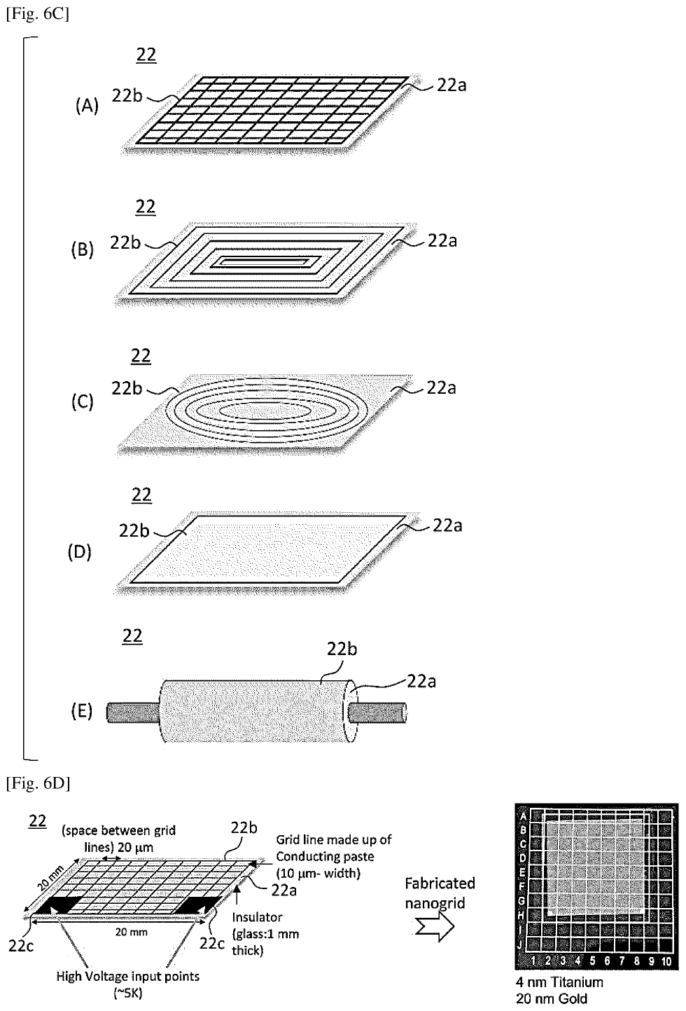

The present disclosure further provides a system for fabricating a fibrous assembly. FIG. 6A shows the overall structure of the system according to an embodiment. In an embodiment, as shown in FIG. 6A, the system 20 includes: a nozzle 21 configured to be supplied with a spinning liquid; and a collector 22 arranged at a distance from the nozzle 21. In a preferable embodiment, example of the spinning liquid includes, for example, but not limited to, a biocompatible polymer (preferably biodegradable biocompatible polymer, more preferably, natural biodegradable biocompatible polymer, selected from the group consisting of, for example, PHA, PHB, or poly (3-hydroxy butyrate)), chitosan, pectin, gelatin, agar-agar, polycaprolactone and collagen, and the polymer optionally further loaded with drugs, neuro hormones or neuro peptides such as serotonin (5-HT), and/or other hormones or peptides such as melanin {for example, the spinning liquid as mentioned above can be supplied to the nozzle 21}.

The nozzle 21 and the collector 22 are located inside a spinning chamber 27. The chamber 27 may be provided with at least one of a humidity controller 27a for measuring the humidity inside the chamber 27 and a temperature sensor 27b for measuring the temperature inside the chamber 27, such that the humidity and/or temperature inside the chamber 27 can be monitored when the spinning liquid is being discharged from the nozzle 21. The chamber 27 may also be provided with an exhaust unit 27c (e.g., a fan) for exhausting the inside of the chamber 27.

In an embodiment, as shown in FIG. 6A, the collector 22 includes a non-conductive base 22a and a conductive target electrode 22b with a pattern, that is disposed on the base 22a. In the embodiment shown in FIG. 6A, the base 22a has a flat plate shape. In a preferable embodiment, an insulating material such as quartz glass can be used as the material of the base 22a. The dimensions of the base 22a may be, for example, but not limited to, 20 mm×20 mm×1 mm.

In the embodiment shown in FIGS. 6A and 6C (A), the target electrode 22b has a grid pattern. In a preferable embodiment, a conductive material such as gold can be used as the material of the target electrode 22b. The target electrode 22b can be formed on the base 22a by known techniques such as imprinting (nano-imprinting), printing, or etching. The line width of the grid lines of the target electrode 22b may be, for example, 20 μm or less, 10 μm or less, or 8 μm or less, 5 μm or less. The interval between two adjacent grid lines may be, for example, 1 μm to 40 μm, 1 μm to 20 μm, or 1 μm to 10 μm.

In an embodiment, as shown in FIG. 6D, the target electrode 22b may be provided with a voltage input points 22c. The target electrode 22b can be electrically connected to a high voltage power supply 28 through the voltage input points 22c, and a high voltage of more than 5 kV, for example, 10 kV to 30 kV can be applied between the nozzle 21 and the target electrode 22b.

In alternative embodiments, the target electrodes 22b may have a concentric rectangular pattern (see FIG. 6C (B)), a concentric circular pattern (see FIG. 6C (C)), or a solid planar shape (see FIG. 6C (D)).

In an embodiment, the collector 22 is an exchangeable or removable collector. The collector 22 is exchangeably or removably mounted and supported on a stage or mount 26 located inside the chamber 27. The collector 22 may be, for example, but not limited to, detachably fixed to the stage or mount 26 by clips 26a. By exchanging the collector 22 to vary the pattern of the target electrode 22b, the arrangement, diameter, orientation and spacing of fibers in a fibrous assembly deposited on the collector 22 can be controlled. In an alternative embodiment, the collector 22 may be a non-exchangeable collector or non-removable collector. The stage or mount 26 may be electrically grounded.

The nozzle 21 is made of conductive material, for example metal and is arranged above the collector 22 and spaced apart from the collector 22. The tip of the nozzle 21 is directed vertically downward and is coated with an insulator. A metal syringe 21a storing the spinning liquid is fixed to the body of the nozzle 21. A syringe pump 21b for controlling flow rate of the spinning liquid supplied to the nozzle 21 is connected to the syringe 21a. The flow rate of the spinning liquid supplied to the nozzle 21 may be, for example, 0.01 to 1 ml/h. The nozzle 21 is electrically connected to the high voltage power supply 28 through the syringe 21a, and a high voltage of more than 5 kV, for example, 10 kV to 30 kV can be applied between the nozzle 21 and the target electrode 22b.

When the high voltage is applied between the nozzle 21 and the target electrode 22b while the spinning liquid is supplied to the nozzle 21, the spinning liquid is electrically charged, and discharged in an electrically charged state toward the target electrode 22b. The discharged spinning liquid is stretched by the repulsive force of electric charges and the solvent in the liquid evaporates to form fibers (for example, nano fibers), and the formed fibers are deposited as fibrous assembly on the collector 22.

In a preferable embodiment, the stage or mount 26 is provided with a moving mechanism 25 configured to move the collector 22 inside the chamber 27. The moving mechanism 25 may be configured to move the collector 22 on the stage or mount 26 in the X and Y directions (horizontal directions), both of which are perpendicular to the discharge direction of the nozzle 21, or in the Z direction (vertical direction) parallel to the discharge direction of the nozzle 21. The moving mechanism 25 may include a piezo motor, such that the amount of movement of the collector 22 can be controlled with a precision of 200 nm or less.

In an alternative embodiment, both the base 22a and the target electrode 22b may have a cylindrical shape (see FIGS. 6B and 6C (E)). In this case, one axial end and the other axial end of the cylindrical collector 22 may be rotatably and exchangeably or removably attached to and supported by a pair of support columns 29 installed inside the chamber 27. The columns 29 are provided with a rotary motor 29a for rotating the cylindrical collector 22 around the central axis thereof. The rotational speed of the collector 22 may be, for example, 0 to 4000 rpm/min. The columns 29 may be electrically grounded.

In a preferable embodiment, the columns 26a are provided with a moving mechanism (not shown) configured to move the cylindrical collector 22 inside the chamber 27 in the X and Y directions. The moving mechanism may include a piezo motor, such that the amount of movement of the cylindrical collector 22 can be controlled with a precision of 200 nm or less.

In a preferable embodiment, the system 20 further includes a control unit 30 for controlling the operation of the moving mechanism 25. At least part of the control unit 30 may be implemented by a computer having a memory storing instructions and at least one processor configured to execute the instructions.

The control unit 30 may include: a data reception unit configured to receive three-dimensional CAD data (for example, an STL file) of a fibrous assembly to be manufactured; a calculation unit configured to calculate timing and amount of movement of the collector 22 according to the CAD data; and a signal transmitting unit configured to transmit a control signal corresponding to the calculated timing and amount of movement to the moving mechanism 25. During discharge of the spinning liquid, the moving mechanism 25 moves the collector 22 according to the control signal received from the control unit 30, thereby controlling the geometrical shape of the fibrous assembly deposited on the collector 22 to a desired shape defined in the CAD data.

The present disclosure further provides a method for fabricating a fibrous assembly using the system 20 as mentioned above.

In an embodiment, the method includes determining the pattern of the target electrode 22b on the collector based on a desired alignment, diameter, orientation and inter-fiber space of fibers in a fibrous assembly. For example, a relationship between the pattern of the target electrode 22b on the collector 22 and the arrangement, diameter, orientation and spacing of fibers of a fibrous assembly deposited on the collector 22 may be determined in advance through experiments and simulations, and the determined relationship is stored in a table or database. Then, the pattern of the target electrodes 22b on the collector 22 may be determined by reference to said table or database based on the alignment, diameter, orientation and inter-fiber space of fibers in a fibrous assembly to be manufactured. In an alternative example, the pattern of the target electrode 22b on the collector 22 may be determined based on the alignment, diameter, orientation, and inter-fiber space of fibers in the fibrous assembly to be manufactured, using a machine-learned model having trained the relationship between the pattern of the target electrode 22b on the collector 22 and the alignment, diameter, orientation, and inter-fiber space of fibers in the fibrous assembly deposited on said collector 22.

In an embodiment, the method further includes arranging the collector 22 having the target electrode 22b with the determined pattern at a distance from the nozzle 21 configured to be supplied with the spinning liquid.

In an embodiment, the method further includes applying a high voltage between the nozzle 21 and the target electrode 22b and electrically charging the spinning liquid, so as for the spinning liquid to be discharged in an electrically charged state toward the target electrode 22b to form fibers, which are deposited as a fibrous assembly on the collector 22. During discharge of the spinning liquid, the moving mechanism 25 may move the collector 22 according to the control signal received from the control unit 30, thereby controlling the shape of the fibrous assembly deposited on the collector 22 to have a desired shape.

In the embodiment mentioned above, the nozzle 21 is stationary positioned inside the chamber 27, and the moving mechanism 25 is configured to move the collector 22 inside the chamber 27 in the X, Y and Z directions, respectively. In an alternative embodiment, the collector 22 may be stationarily positioned inside the chamber 27 and the moving mechanism may be configured to move the nozzle 21 inside the chamber 27 in the X, Y and Z directions, respectively. In another alternative embodiment, the moving mechanism may be configured to move both the nozzle 21 and the collector 22 independently of each other inside the chamber 27 in the X, Y and Z directions, respectively.

In alternative embodiment, the system 20 may include a plurality of the nozzles 25. In a preferable embodiment, the plurality of nozzles 25 may each have a different nozzle diameter. In this case, by simultaneously discharging the spinning liquids from the plurality of nozzles 25, the fibrous assembly can contain a plurality of types of fibers each having different diameters.

In a preferable embodiment, the plurality of nozzles 25 may each be supplied with a different spinning liquid or a combination of a spinning liquid, a gas and a solvent. In this case, by simultaneously discharging the spinning liquids or combinations from the plurality of nozzles 25, the fibrous assembly can contain a plurality of types of fibers each having different properties.

In an embodiment, the present disclosure provides, for example, the following inventions:

[1] A fiber (in particular, a biocompatible fiber, a nano fiber, or a biocompatible nano fiber), comprising a biocompatible polymer and melanin, wherein the polymer may preferably be a biodegradable polymer, more preferably be a natural biodegradable polymer, or a fibrous assembly comprising the fiber, wherein the fiber or the fibrous assembly may be dried (e.g., lyophilized), for example, a fiber, comprising a biocompatible polymer and melanin, or a fibrous assembly comprising the fiber . . .

[2] The fiber or fibrous assembly according to [1] above, wherein the polymer is a biodegradable polymer.

[3] The fiber or fibrous assembly according to [1] or [2] above, further comprising serotonin (5-HT).

[4] The fiber or fibrous assembly according to any one of [1] to [3] above, wherein the biocompatible polymer is polyhydroxy alkane (PHA), preferably polyhydroxy butyrate (PHB), more preferably poly (3-hydroxy butyrate).

[5] The fiber or fibrous assembly according to any one of [1] to [4] above, having a conductivity of 1×10² to 1×10⁵ S/cm.

[6] The fiber or fibrous assembly according to any one of [1] to [5] above, having a hydrophilic surface (e.g., exhibiting a contact angle of 55 degree or more to less than 90 degree).

[7] The fiber or fibrous assembly according to any one of [1] to [6] above, wherein the fiber is coated with laminin and optionally poly L-lysine.

[8] The fiber or fibrous assembly according to any one of [1] to [7] above, wherein the fiber satisfies at least one of:
(i) having 100 nm to 2 μm (e.g., 250 nm to 1.5 μm) in diameter;
(ii) having a crystallization temperature of 160° C. to 190° C.;
(iii) having a Young's modulus of 10 MPa to 90 MPa;
(iv) exhibiting a contact angle of less than 90° (e.g., 55° to 80° C.); and
(v) exhibiting a high biodegradability compared to a fiber that contains no melanin and no serotonin.

[8A] The fiber or fibrous assembly according to any one of [1] to [7] above, wherein the fiber satisfies at least one of:
(i) having 100 nm to 2 μm (e.g., 250 nm to 1.5 μm) in diameter;
(ii) having a crystallization temperature of 160° C. to 190° C.;
(iii) having a Young's modulus of 10 MPa to 90 MPa;
(iv) exhibiting a contact angle of less than 90° (e.g., 55° to 80° C.);
(v) having a conductivity of 1×10² S/cm to 1×10⁵ S/cm, 1×10² S/cm to 1×10+S/cm, or 1×10² S/cm to 1×10³ S/cm; and
(vi) exhibiting a high biodegradability compared to a fiber that contains no melanin and no serotonin.

[9] A scaffold (preferably an implantable scaffold) or scaffold material, implantable material or implant, comprising a plurality of the fibers or fibrous assembly according to any one of [1] to [8A] above (hereinafter [1] to [8A] includes [8] above), wherein the fibers may be aligned in a reticular manner in the scaffold or scaffold material, implantable material or implant.

[10] The scaffold (preferably an implantable scaffold) or scaffold material, implantable material or implant according to [8] above, for use in inducing nerve regeneration, wherein the implantable scaffold or scaffold material, implantable material or implant is optionally cut, folded, or rolled (e.g., in brain, spinal cord, or peripheral nerve).

[11] A method of transplanting a graft material in a subject (e.g., human subject) in need thereof, comprising:
transplanting the graft material to the subject,
wherein the graft material comprises the fiber or fibrous assembly, scaffold (preferably an implantable scaffold) or scaffold material, implantable material or implant,
comprising a plurality of the fibers according to any one of [1] to [8A] above; wherein
a plurality of the fibers may have an aligned pattern and/or preferably be aligned in a reticular manner in the graft material; and wherein the graft material may further comprise a nerve cell for nerve regeneration on a surface of the graft,
thereby optionally inducing nerve regeneration in the subject.

[12] The method according to above, wherein the graft material further comprises a nerve cell for nerve regeneration on a surface of the graft, thereby inducing nerve regeneration in the subject.

In an embodiment, the present disclosure further provides, for example, the following inventions:

[9] A System for Fabricating a Fibrous Assembly Comprising:
a nozzle configured to be supplied with a spinning liquid; and
a collector arranged at a distance from the nozzle, wherein
the collector includes a target electrode with a pattern and when a voltage is applied between the nozzle and the target electrode, the spinning liquid is discharged in an electrically charged state toward the target electrode to form fibers, which are deposited as a fibrous assembly on the collector, such that an alignment, diameter, orientation and inter-fiber space of the fibers in the fibrous assembly can be controlled by varying the pattern of the target electrode.

[13A] A system for fabricating a fibrous assembly comprising:
a nozzle configured to be supplied with a spinning liquid; and
a collector arranged at a distance from the nozzle, wherein
the collector includes a target electrode with line shape/circular shape arranged in a predetermined geometry and pattern and when voltage is applied between the nozzle and the target electrode, the charged spinning liquid is discharged toward the target electrode to form fibers, which are deposited as a fibrous assembly on the collector, so that an alignment, diameter, orientation and inter-fiber space of the fibers in the fibrous assembly may be controlled by varying the geometry and pattern of the target electrode.

[14] The system according to or [13A] above, further comprising a moving mechanism configured to move one or both of the nozzle and the collector relative to each other, such that a shape and size of the fibrous assembly can be controlled by moving the nozzle and the collector relative to each other during discharge of the spinning liquid.

[14A] The system according to or [13A] above, further comprising a moving mechanism configured to move one or both of the nozzle and the collector relative to each other, so that a shape and size of the fibrous assembly may be controlled by moving the nozzle and the collector relative to each other during discharge of the spinning liquid.

[15] The system according to or [14A] above, wherein
the moving mechanism is configured to move one or both of the nozzle and the collector relative to each other in the X direction and the Y direction, both of which are perpendicular to the discharge direction of the nozzle.

[15A] The system according to or [14A] above, wherein
the moving mechanism is configured to move one or both of the nozzle and the collector relative to each other in X direction and Y direction perpendicular to the discharge direction of the nozzle.

[16] The system according to any one of to [15A] above (hereinafter to [15A] includes [14A] and above), wherein
the moving mechanism is configured to move one or both of the nozzle and the collector relative to each other in the Z direction parallel to the discharge direction of the nozzle.

[16] The system according to any one of to above (hereinafter to includes [14A], and [15A] above), wherein
the moving mechanism includes a piezo motor, such that the amount of movement of one or both of the nozzle and the collector can be controlled with a precision of 200 nm or less.

[17A] The system according to any one of to above, wherein
the moving mechanism includes a piezo motor, so that the amount of movement of one or both of the nozzle and the collector may be controlled with a precision of 200 nm or less.

[18] The system according to any one of to [17A] above (hereinafter to [17A] includes [14A], [15], [15A], and above), further comprising a control unit
configured to control an operation of the moving mechanism, wherein the control unit includes
a data reception unit configured to receive CAD data of a fibrous assembly to be manufactured,
a calculation unit configured to calculate timing and amount of movement of one or both of the nozzle and the collector according to the CAD data, and
a signal transmitting unit configured to transmit control signal according to the calculated timing and amount of movement to the moving mechanism.

[19] The system according to any one of to above (hereinafter to includes [13A], [14], [14A], [15], [15A], [16], and [17A] above), comprising a plurality of the nozzles.

[20] The system according to above, wherein
the plurality of nozzles each have a different nozzle diameter, such that the fibrous assembly can contain a plurality of types of fibers with different diameters by simul-tancously discharging the spinning liquids from the plurality of nozzles.

[20A] The system according to above, wherein
the plurality of nozzles have different nozzle diameters, so that the fibrous assembly may contain a plurality of types of fibers with different diameters by simultaneously discharging the spinning liquids from the plurality of nozzles.

[21] The system according to any one of to [20A] above (hereinafter to [20A] includes above), wherein
the plurality of nozzles each are supplied with a different spinning liquid or a combination of a spinning liquid, a gas and a solvent, such that the fibrous assembly can contain a plurality of types of fibers with different properties by simultaneously discharging the spinning liquids or combinations from the plurality of nozzles.

[21A] The system according to any one of to [20A] above, wherein
the plurality of nozzles are supplied with different spinning liquids or combinations of spinning liquids, gases and solvents, so that the fibrous assembly may contain a plurality of types of fibers with different properties by simultaneously discharging the spinning liquids or combinations from the plurality of nozzles.

[22] The system according to any one of to [21A] above (hereinafter to [21A] includes [13A], [14], [14A], [15], [15A], [16], [17], [17A], [18], [19], [20], [20A] and [21] above), further comprising: a spinning chamber the nozzle and the collector are located therein; and at least one of a humidity controller and a temperature sensor inside the spinning chamber.

[22A] The system according to any one of to [21A] above, further comprising at least one of a humidity controller and a temperature sensor inside a spinning chamber.

[23] The system according to any one of to [22A] above (hereinafter to [22A] includes [13A], [14], [14A], [15], [15A], [16], [17], [17A], [18], [19], [20], [20A], [21], [21A] and above), wherein the collector has a grid pattern, a concentric rectangular pattern, a concentric circular pattern, a solid planar shape, or a cylindrical shape.

[24] The system according to any one of to above (hereinafter to includes [13A], [14], [14A], [15], [15A], [16], [17], [17A], [18], [19], [20], [20A], [21], [21A], and [22A] above), wherein the collector is an exchangeable or removable collector.

[25] A collector with a target electrode for use in the system according to any one of to above (hereinafter to includes [13A], [14], [14A], [15], [15A], [16], [17], [17A], [18], [19], [20], [20A], [21], [21A], [22], [22A] and above).

[26] A method for fabricating a fibrous assembly comprising:
determining a pattern of a target electrode on a collector based on a desired alignment, diameter, orientation and inter-fiber space of fibers in a fibrous assembly;
arranging the collector conducting in nature and having the target electrode at a distance from a nozzle configured to be supplied with a spinning liquid; and
applying a voltage between the nozzle and the target electrode, so as for the spinning liquid to be discharged in an electrically charged state toward the target electrode to form fibers, which are deposited as the fibrous assembly on the collector.

[26A] A method for fabricating a fibrous assembly comprising:
arranging a collector conducting in nature and having a target electrode with line shape/or circular shape arranged in a predetermined geometry and pattern at a distance from a nozzle configured to be supplied with a spinning liquid; and
applying a voltage between the nozzle and the target electrode, so that the charged spinning liquid is discharged toward the target electrode to form fibers, which are deposited as a fibrous assembly on the collector, wherein an alignment, diameter, orientation and inter-fiber space of the fibers in the fibrous assembly may be controlled by varying the geometry and pattern of the target electrode.

[27] A method of producing a fiber, comprising:
arranging a collector having a target electrode at a distance from a nozzle configured to be supplied with a spinning liquid; and
applying a voltage between the nozzle and the target electrode, so as for the spinning liquid to be discharged in an electrically charged state toward the target electrode to form fibers, wherein the spinning liquid comprises a biocompatible polymer (preferably biodegradable biocompatible polymer, more preferably, natural biodegradable biocompatible polymer, selected from the group consisting of, for example, PHA, PHB, or poly (3-hydroxy butyrate)), chitosan, pectin, gelatin, agar-agar, polycaprolactone and collagen, and the polymer optionally further loaded with drugs, neuro hormones or neuro peptides such as serotonin (5-HT), and/or other hormones or peptides such as melanin {for example, the fiber according to any one of [1] to [8A] above can be obtainable by the method}.

[27A] A method of producing a fiber, comprising:
arranging a collector having a target electrode at a distance from a nozzle configured to be supplied with a spinning liquid; and
applying a voltage between the nozzle and the target electrode, so that the charged spinning liquid is discharged toward the target electrode to form fibers, which are deposited as a fiber(s) on the collector, wherein the spinning liquid comprises a biocompatible polymer (preferably biodegradable biocompatible polymer, more preferably, natural biodegradable biocompatible polymer, selected from the group consisting of, for example, PHA, PHB, or poly (3-hydroxy butyrate)), chitosan, pectin, gelatin, agar-agar, polycaprolactone and collagen, and the polymer optionally further loaded with drugs, neuro hormones or neuro peptides such as serotonin (5-HT), and/or other hormones or peptides such as melanin {for example, the fiber according to any one of [1] to [8A] above can be obtainable by the method}.

[28] A semiconductor device such as a transistor, an implant, or an electrode, comprising the fiber or fibrous assembly according to any one of [1] to [8A] above having a conductivity of 1×10² to 1×10⁵ S/cm.

### EXAMPLE

1. Materials and methods

### 1.1 Reagents and Antibodies

Synthetic Melanin (Sigma M8631-1G), Poly [(R)-3-hydroxybutyric acid (Sigma 363502-100G), and Serotonin hydrochloride (Sigma H9523-1G), 1, 1, 1, 3, 3, 3-Hexafluoro-2-propanol (HFIP) (Sigma-Aldrich, St. Louis, USA), poly (vinyl alcohol) 8.0 wt % (Kato tech, Japan) were used for the fabrication of scaffolds. All chemicals were supplied by Sigma Aldrich, unless stated otherwise. SYLGARD™ 184 silicon elastomer kit (Dow chemical company, USA) was used to prepared cured PDMS ring for the culture chamber. Suppliers for tissue culture media and sup-plements are individually specified in the method section. 16% paraformaldehyde (#15710; Electron Microscopy Sciences, Hatfield, PA) was purchased from the for the fixation of the cells. Anti-BIII tubulin antibody was purchased from GeneTex (#GTX631830) and AlexaFluor 594 anti-mouse secondary antibody was purchased from Invitrogen (ThermoFisher Scientific, Japan).

### 1.2. Preparation of Electrospun Fibers

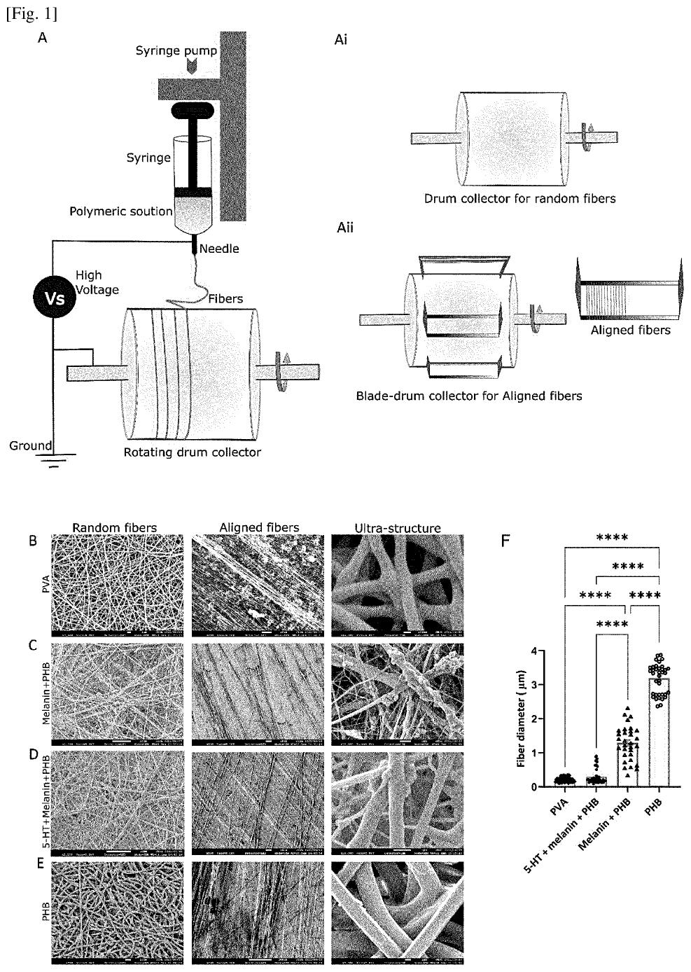

PHB was used as a blend to assist electrospinning (5-HT/melanin/PHB and melanin/PHB). The electrospinning setup used in the preparation of random and aligned fibers is shown in FIG. 1A. Solutions of PHB (9% w/v) and 5-HT (1.5 mM) together with melanin (3% w/v) were prepared in hexafluoro-2-proopanol (HFIP). The PHB solution was heated mildly in a hot water bath and the resultant clear solution was stirred overnight for proper mixing. Later, solution of 5-HT and melanin were added to the PHB solution and stirred for 4 h at RT and subsequently used for the electrospinning (FIG. 1A). The homogenously mixed polymer solution was poured in a glass syringe with 22-gauge flat tip needle to prevent point discharge effects. For the fabrication of random fibrous sheets, polymer solution was ejected using a syringe pump at a flow rate of 0.022 mm/min towards the rotating cylindrical target (5.42 m/min) (FIG. 1Ai), under high-electric potential of 12 kV. the traverse speed of syringe (left to right movement) was 5.14 cm/min and the distance between the tip of the needle and the cylindrical collector was set at 10 cm. Similarly, for the fabrication of aligned fibrous sheets, polymer solution was ejected using a syringe pump at a flow rate of 0.035 mm/min towards rectangular blades fixed on rotating drum target (2 m/min) (FIG. 1Aii), under high-electric potential (10 k). The traverse speed of syringe (left to right movement) was 4.15 cm/min and the distance between the tip of the needle and the cylindrical collector was set at 8 cm. In both conditions (random/aligned); relative humidity (48%) and temperature (25° C.) were kept constant during electrospinning. Fibers were collected and dried overnight in vacuum desiccator to remove the solvent.

### 1.3. Characterization:

### 1.3.1 Surface Morphology

Surface morphology of PHB, melanin/PHB, 5-HT/melanin/PHB and PVA fibers was evaluated using scanning electron microscopy (SEM, JEOL JSM-7900F). Briefly, electrospun fibers were lyophilized and mounted onto a conducting carbon tape attached on a copper stub. Samples were sputter coated with gold for 4 min followed by analysis under SEM at a working distance of 10 mm and an accelerating voltage of 5 kV. The surface morphology and ultra-structure of the fibers was observed at various magnifications ranging from 500X to >20000X. The average and standard error of the mean of the diameter of electrospun fibers was determined from the SEM images.

### 1.3.2. Physiochemical and Electrical Properties

### 1.3.2.1. Chemical Composition/Functional Group Analysis

Functional group presence on electrospun nanofibers and powder forms of PHB, melanin and 5-HT (with KBr) were performed with a Fourier-transform infrared spectroscopy (FTIR) machine (Vertex 80v; Bruker) in transmission mode over a range of 400-4000 cm⁻¹ (MIR range) at the resolution 2 cm/4 cm⁻¹ with 64 scans averaging mode. Further, elemental analysis (carbon, oxygen and nitrogen) was performed using X-ray photoelectron spectroscopy (XPS) on a KRATOS Axis Ultra has (S L V Narayana et al., 2020).

### 1.3.2.2 Mapping the Elemental Composition of Fiber

Tracing of the fibers' elemental composition using SEM-EDX was also performed to confirm successful blending of melanin and PHB and map the distribution of melanin in blended fibers. Briefly, scaffolds were sputter coated with Osmium (Os) for 2 min and visualized using a scanning electron microscope (SEM; JEOL JSM-7900F), equipped with a front and rear Oxford energy-dispersive X-ray (EDX) detector (Oxford Instruments, UK). SEM micrographs were captured at a working distance of 10 mm and an accelerating voltage of 10 kV at 500x. Aztec-SEM 6.0 (Oxford Instruments, UK) software was used to map the distribution of C, O and N elements in PHB and blended 5-HT-melanin-PHB (9% PHB-3% melanin) fibers.

### 1.3.2.3. Thermal Properties

Differential scanning calorimetry (DSC)-8500 (PerkinElmer) was used for the thermal analysis. Scaffold samples of 2 mg were placed in alumina pans and empty pans were used as a reference. All samples were first heated at a range of 40 to 250° C. with a rate of 10° C./min. Afterwards, the samples were cooled to 40° C. at 10° C./min. After each test, the crystallization/melting peak region from the thermograph was analysed to determine the crystallization (Tc)/melting (Tm) point (Agrawal et al., 2021, 2022).

### 1.3.2.4. Mechanical Properties of Electrospun Fibers

Atomic Force Microscope (AFM) images of the scaffolds were acquired as previously described (Agrawal et al., 2021, 2022), using a MultiMode 8 Atomic Force Microscope with a Nanoscope V controller and E scanner (Bruker). Mechanical characterization of the scaffold nanofibers was performed using a PeakForceTapping mode. AFM imaging was conducted with RTESPA-150 probe (Bruker) with a nominal spring constant of 5.1 N/m, nominal frequency of 2 kHz and a nominal tip radius of 15 nm (Baklaushev et al., 2019). Nanogrid scaffolds were studied in air over the area of 5×5 μm to determine force-displacement curves. The reduced Young's Modulus/DMT modulus, is automatically calculated by fitting the retract curve using the Derjaguin, Muller, Toropov (DMT) model (Lagaly, 1988). Images were obtained at a scan rate of 1 Hz and 512×512 pixels' resolution. The raw Young's modulus AFM images were processed using the NanoScope Analysis v.1.10 software (Bruker).

### 1.3.2.5. Electrical Properties of Electrospun Fibers

Conductivity of the electrospun fibers mesh of 5-HT/melanin/PHB (80 μm thickness) and melanin/PHB (60 μm) under dry condition was determined by using a probe station Summit 12K(Cascade Wernersville, PA, USA) equipped with a Keithley SC 4200A parameters analyser (Tektronix, Oregon, USA). We used van der Pauw method for resistivity measurement (4-probes method in which two pairs of contacts are used to measure the conductivity).

### 1.4. Surface Wettability Measurement

The wettability of the scaffold was evaluated by static contact angle measurement using an Easy Drop tensiometer (KRUSS, GmbH) (Agrawal et al., 2021). The contact angle is a quantitative measure of the wetting properties of a solid by liquid and is dependent on the surface area, with higher surface energies being associated with lower contact angles (Morouco et al., 2016). A water droplet was poured onto the surface of solid samples and the contact angle was measured by Drop Shape analysis software (KRUSS, GmbH).

### 1.5. Degradation Profile

Electrospun fibrous meshes swelling and degradation in phosphate buffered saline (PBS, pH 7.4) was monitored. Briefly, dried electrospun fibers scaffolds were cut into 10 mm×10 mm samples. Subsequently, samples were placed in a 24-well plate containing 1 ml of PBS (pH 7.4) and incubated in a water bath shaker (30 strokes/min) at 37° C. (Agrawal et al., 2022). To calculate the degree of swelling, samples were immersed in 1 ml of PBS (pH 7.4) at 37° C. for 12 h, and subsequently weighed. AFM micrographs were also taken to visually confirm the swelling of fibers. Similarly, for the degradation analysis, after the pre-determined degradation period (10 days), the scaffolds were rinsed in PBS and dried in an oven for 24 h and weighted. For the calculation of the degree of swelling (%) and degraded mass (%), we considered the initial dry weight of the scaffold (mi), the weight of the swelled nanofibers after removing excess water and surface moisture with a filter paper (ms), and the constant residual weight of the scaffolds after degradation (mx). The degree of swelling and degraded mass (%) of the scaffolds were calculated from equation (5) and (6).
Degree of swelling=(ms-mx/mx)*100 (5)
Degraded mass (%)=(mi-mx/mi)*100 (6)

### 1.6. Biocompatibility Assay

### 1.6.1 Ethics Statement

All experiments were carried out following the guidelines of the Okinawa Institute of Science and Technology Graduate University (OIST) genetic manipulation procedures. All animal experiments have been performed in the accordance to Japanese laws and to the regulations of the OIST animal care and use committee (protocol #ACUP-2021-326) OIST animal facilities and animal care and use program are accredited by AAALAC international (Ref. #1551).

### 1.6.2. Preparation for In Vitro Culture

We used plastic bottom 24 well plate for primary neural cell culture. Scaffolds were placed inside the well and a Polydimethylsiloxane (PDMS) ring was inserted to fix the scaffold at the bottom of the well. For the fabrication of Polydimethylsiloxane (PDMS) rings, molds were designed in CAD Rhinoccrous3D (V.5, Robert McNeel & Associates) and 3D printed (Object 500 Connex 3, Stratasys, Germany). PDMS prc-polymer and catalyst were then mixed thoroughly with a 10:1 ratio in a disposable plastic cup. The mixture was degassed in a vacuum desiccator for 20 minutes and poured inside the molds. The PDMS was cured in an oven at 60° C. for 3 h. Finally, polymerized PDMS rings were extracted from the molds and fixed on the scaffold to secure it at the bottom of the well for neuronal culture.

### 1.6.3. Culture of Dorsal Root Ganglionic (DRG)/Sensory Neurons

The structure was then coated with 0.01% Poly-L-lysine (Sigma-Aldrich #P4832-50 ml) overnight at 4° C., rinsed with water and coated again with Laminin (GibcoBRL #23017-015) for 2 h at 4° C. DRGs were dissected from 2 months old ICR female mice (Charles River or Japan Clea, Japan) and dissociated as previously described (Agrawal et al., 2021; Ben-Yaakov et al., 2012; Terenzio et al., 2018). Briefly, after dissection, DRGs were dissociated by sequential digestion with 100 U of papain (Sigma-Aldrich, #P4762) in HBSS (GibcoBRL, #14175095), followed by digestion with 1 mg/ml collagenase-II(Worthington Biochemical Corporation, #CLS2) and 1.2 mg/ml dispase at 37° C. in HBSS for at least 30 minutes. The ganglia were then triturated in HBSS, 10 mM Glucose, and 5 mM HEPES Sigma-Aldrich, #H0887), pH 7.35. Cells were recovered by centrifugation through 20% Percoll (Sigma-Aldrich, #P4937) in L15 medium (GibcoBRL #L-5520) at the speed of 1000 rpm for 7 min, plated at a density of 2×10+ cells/scaffold and grown in F12 medium (GibcoBRL #11765062) for 48 hours/DIV-2.

1.6.4. Culture of hMNs from IPS Cells

We adapted a published protocol (Bossolasco et al., 2018) for the culture and differ-entiation of human inducible pluripotent stem cells (hIPSCs) into motor neurons (MNs). Human MNs were cultured for 7-DIV prior fixation and immunostaining.

### 1.6.5. Immunohistochemistry of Cultured Neurons

DRG neurons were cultured for 2 days and MNs for 7 days as described above before fixation with 4% paraformaldehyde in phosphate buffer saline (PBS) for 30 min at room temperature. Nonspecific antibody binding was blocked by incubation with 2% normal goat serum and 0.1% Triton X-100 in PBS for 30 min. To visualize the axonal network, neurons were incubated overnight at 4° C. with anti-βIII-tubulin antibody (1:1000 dilution in PBS). Cells were then washed 3 times with PBS and incubated with anti-mouse Alexa Fluor 594 conjugated secondary antibody (1:500 dilutions in PBS) for 1 h at room temperature. Fluorescence imaging was performed on a confocal laser scanning microscope LSM900 (Carl Zeiss AG, Germany) using 63x oil immersion objective (Plan-Apochromat DIC M27, NA=1.40). Images were acquired in ZenBlue 3.1 (Carl Zeiss AG) with a 512×512 pixel resolution and a 2.05 ρs pixel dwell time. A 3×3 tiles and z-stacks (with 1 μm steps) image was scanned to encompass the entire grid scaffold on xyz planes. After stitching tiles, the resulting image had a final xy resolution of 1433×1434 pixels.

### 1.7. Statistical Analysis

GraphPad Prism 9 Software, (GraphPad, U.S.A.) was used for statistical analysis. ANOVA with Tukey's post hoc test was performed for multiple comparisons and unpaired t-test was performed for the comparison between 2 groups. Differences were considered significant if the probability of error was less than 5%. All the data were expressed as mean±s.c.m., error bar indicates standard error of mean. * P<0.05, ** P<0.01, *** P<0.001, and **** P<0.0001.

### 2. Results

### 2.1 Surface Characterization, Morphology and Diameter Analysis of Nanofibers

Scanning Electron Microscope (SEM) and Helium Ion Scanning Microscopy (HIM) revealed the ultrastructure of the nanofiber scaffolds (FIG. 1B-1F). Both 5-HT-melanin and PHB composite nanofibres (FIG. 1D) and PHB blended melanin nanofibers (FIG. 1C) have rough surface contrary to PVA and PHB fibers which have smooth surface texture (FIGS. 1B and 1E). Additionally, data showed that both random and aligned fibers were fabricated using electrospun (FIG. 1B-1E; left and middle panel). Direct measurement of fiber diameter with SEM showed that average diameter of the PVA fibers (0.20+0.007 μm) and 5-HT+melanin+PHB fibers (0.29±0.03 μm) is smaller (p ****) than melanin blended PHB fibers (1.28±0.08 μm) and PHB fibers (3.18±0.07 μm) (FIG. 1F).

### 2.2. Physiochemical Properties

The physio-chemical properties scaffolds were investigated by measuring the chemical, thermal, mechanical, electrical properties and hydrophilicity. These properties determine the stability, biocompatibility and biodegradability of materials in vivo and its ability to maintain its intended structure over a period of time to support the growth of cells or tissues.

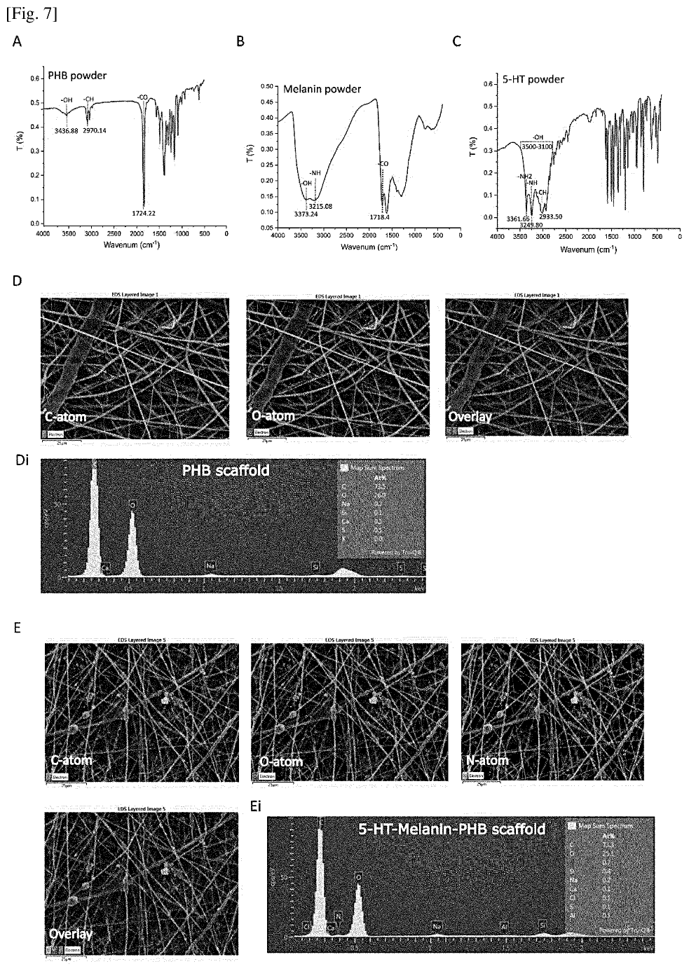

First, we performed chemical analysis of the scaffolds with FT-IR. UV-vis trans-mittance spectra of pure PHB, melanin and 5-HT powders show the presence of characteristic aliphatic-O—H stretching peak at 3436.88 cm⁻¹ (FIG. 7A), aromatic-O—H stretching peak at 3373.24 cm⁻¹ (FIG. 7B) and merged aromatic-O—H peak between 3500-3100 cm⁻¹ (FIG. 7C) respectively. Primary and secondary anime peak were observed in 5-HT sample at 3361.66 cm⁻¹ and 3249.80 cm⁻¹ (FIG. 7C), while we could detect only a secondary amine peak in melanin at 3215.08 cm⁻¹ (FIG. 7B). Further, we observed a sp³-C—H stretching peak in powder PHB and 5-HT at 2970.14 cm⁻¹ and 2933.50 cm⁻¹ respectively (FIGS. 7A and 7C). Carboxylic-C═O stretching peak was observed in PHB and melanin at 1724.22 cm⁻¹ and 1718.44 cm⁻¹ (FIGS. 7A and 7B).

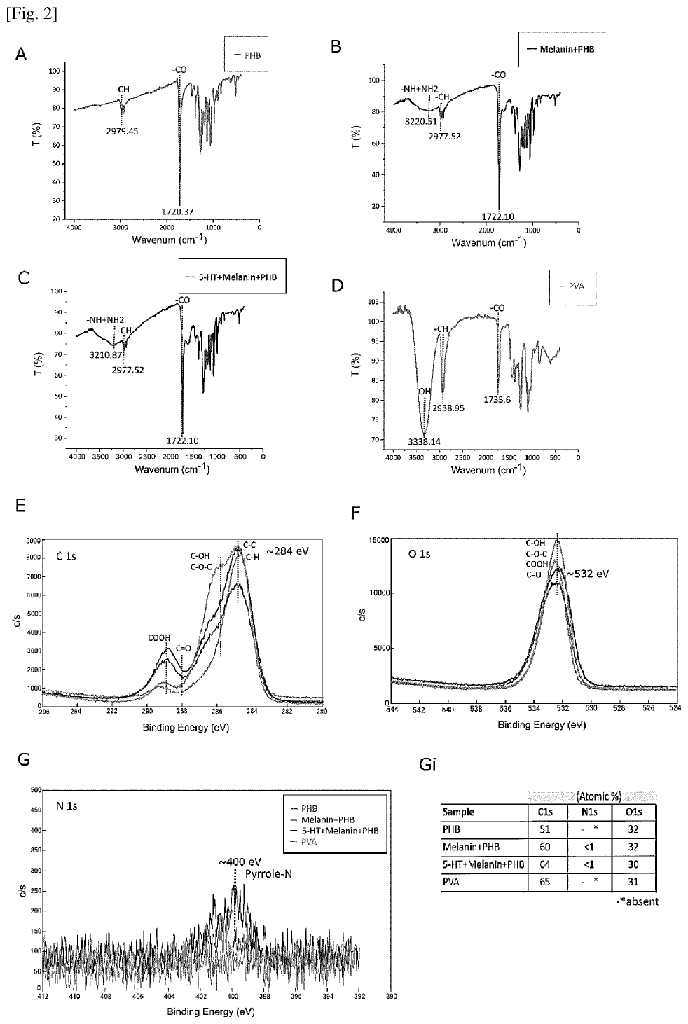

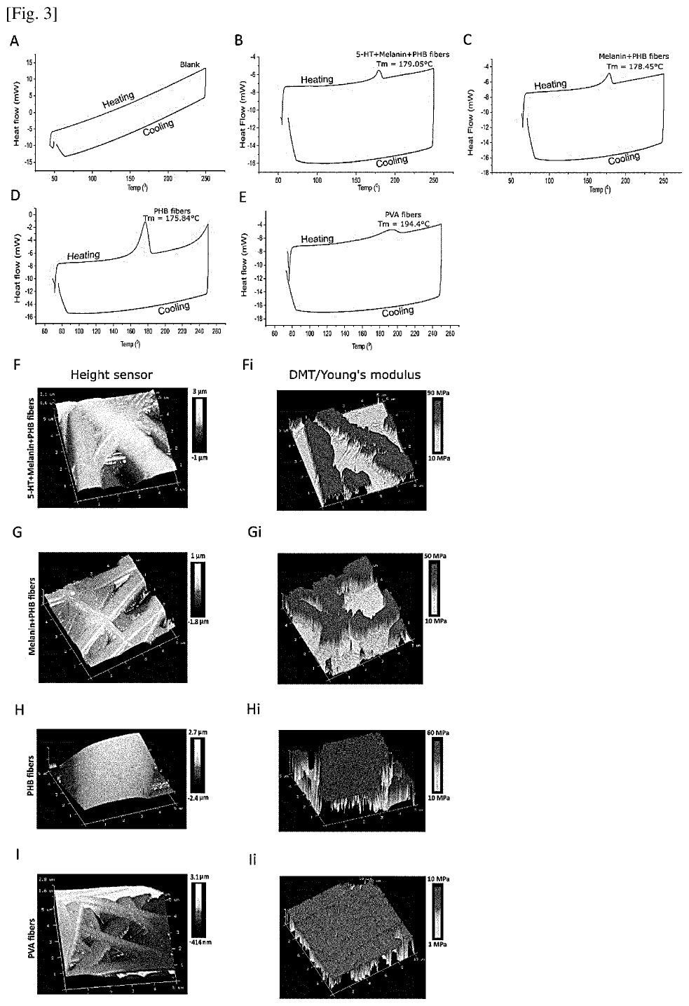

We also characterize the FT-IR spectra for PHB, melanin blended PHB, 5-HT-melaninand PHB composite fibers and PVA fibers. We observed the presence of carboxylic acid-C═O stretching peak at 1720.37 cm⁻¹, sp³-C—H stretching peak at 2979.45 cm⁻¹ in PHB fibers with a shift of ˜4 cm⁻¹ and ˜9 cm⁻¹ with PHB powder form due to the reaction with HFIP(FIG. 2A). A broad primary amine peak at 3220.51 cm, sp³-C—H stretching peak at 2977.52 cm⁻¹ and carboxylic acid-C═O stretching peak at 1722.10 cm⁻¹ in melanin and PHB composite fibers with a significant shift in the peak values with PHB fibers; and melanin powder was also observed (FIG. 2B). Similarly, we identified a broad and structured peak of primary and secondary amine at 3210.87 cm⁻¹, a sp³-C—H stretching peak at 2977.52 cm⁻¹ and carboxylic acid-C═O stretching peak at 1722.10 cm⁻¹ in 5-HT-melanin and PHB composite fibers with a significant shift in the peak values with PHB fibers and 5-HT powder (FIG. 2C). A broad strong peak of —O—H at 3338.14 cm⁻¹, a sp³-CH stretching peak at 2938.95 cm⁻¹ and amide-C═O stretching peak at 1735.60 cm⁻¹ in PVA fibers were observed (FIG. 2D) (Goudappagouda et al., 2020).

Moreover, elemental composition analysis with X-ray photoelectron spectroscopy (XPS) of PHB, melanin+PHB+5-HT, melanin+PHB, and PVA are shown in FIG. 2E-Gi. All the polymers have similar core levels of C Is (FIG. 2E), and O 1s (FIG. 2F), peaks were located at ˜284 cV and ˜532 cV respectively. However, N Is peak located at ˜400.0 cV (FIG. 2G) is present only in melanin-PHB blended fibers and 5-HT-melanin-PHB composite fibers (FIG. 2Gi), which suggest the presence of Pyrrole-N group.

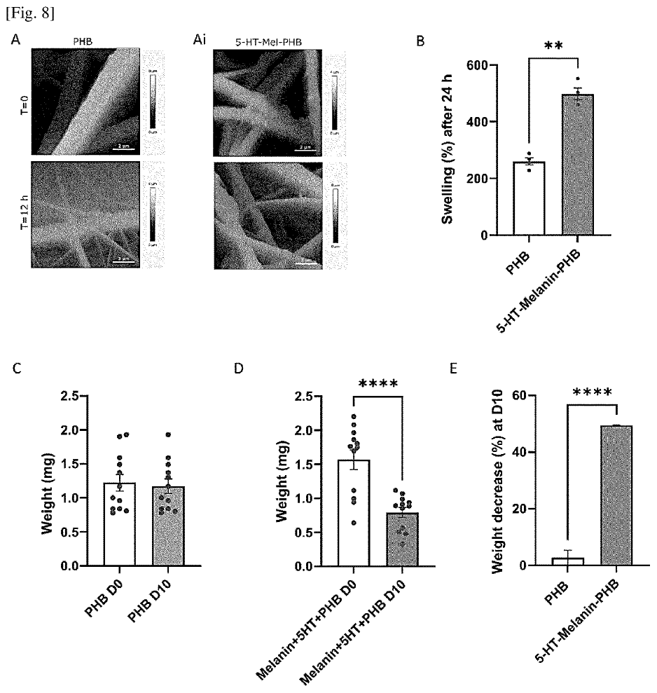

We also traced fiber composition by SEM-EDX analysis (FIG. 7D-E). We focused on the localization of nitrogen to map the distribution of 5-HT and melanin in blended fibers, which is present in both functional groups of 5-HT and melanin and absent in PHB. We compared fibers obtained by electrospinning of PHB (control sample) (FIG. 8D) and a 5-HT-PHB-melanin blend (FIG. 8E). Indeed, the characteristic peak of carbon (Kα 277 cV) and oxygen (Kα 525 eV) was detected in both samples, while nitrogen (Kα 392 cV) was present only in 5-HT-melanin-PHB fibers accounting the 0.7% of nitrogen atoms in the composition of blend (FIG. 8Ei).

We then determined the thermal properties of our scaffolds by DSC (FIG. 3A-E). FIG. 3B-3E shows the negative peak that occurs during heating cycle indicating the melting temperature (Tm) of nanofibers, although there was no peak observed during the cooling cycle, which suggests that, after melting, the polymer remains in the same state (Goudappagouda et al., 2019, 2020). The calculated T. of 5-HT-melanin and PHB composite fibers, melanin blended PHB fibers, PHB fibers and PVA fibers were 179.05° C. (FIG. 3B), 178.45° C. (FIG. 3C), 175.84° C. (FIG. 3D), and 194.4° C. (FIG. 3E), are consistent with thermally stable materials. Thus, our fibers are suitable candidate for being used as an implant or conduit in an in vivo setting.

To measure the mechanical strength of our scaffolds, we used nanoindendation atomic force microscopy (AFM) to generate maps of Young's modulus distribution concurrently with topographical imaging to map the anisotropic distribution of mechanical strength (FIG. 3F-31). The value of the Young's modulus varied in accordance with the structure of the fiber, center of the fiber having a higher value than its periphery (FIG. 3Fi, Gi, Hi, and Ii). The calculated Young's modulus (Y) for the 5-HT a melanin and PHB composite nanofibers (FIG. 3Fi), melanin blended PHB nanofibers (FIG. 3Gi), PHB nanofibers (FIG. 3Hi), and PVA nanofibers (FIG. 3Ii) displayed a range from 10 MPa to 90 MPa, suggesting a considerable potential for the nanofibers to sustain their spatial architecture and mechanical properties and, thus, their suitability as implant for a CNS or soft tissues engineering.

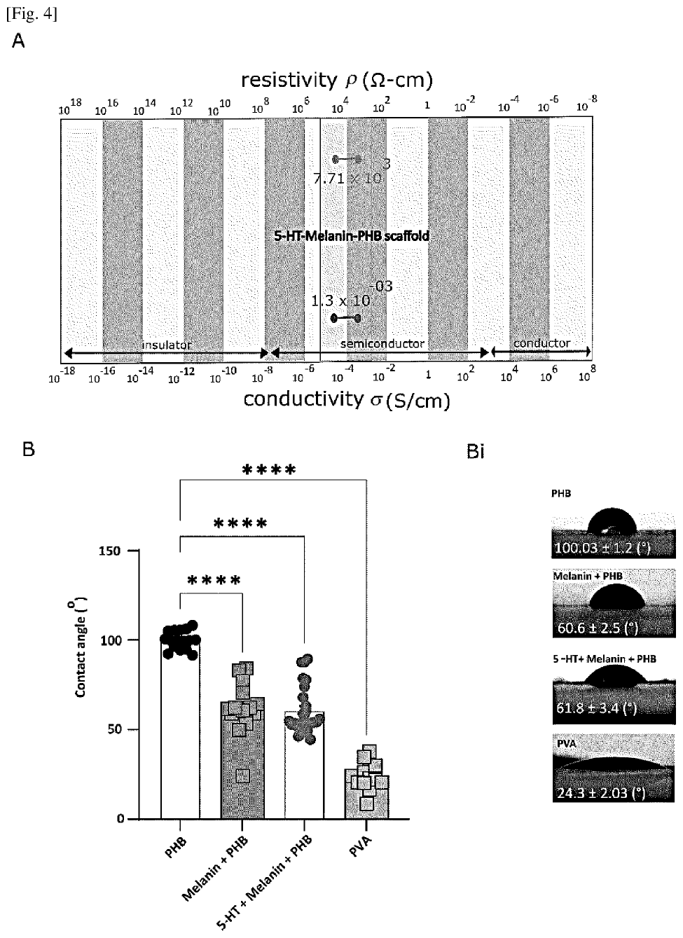

We also measured the resistivity (p) and conductance (o) of the 5-HT and melanin blended scaffold to check the electrical properties of electrospun fibers. Data from the 4-probe method suggested that the resistivity of the fibrous scaffold (7.71×10³ ohm-cm) and conductivity (1.3×10³ S/cm) lies within the range of semiconductor materials (FIG. 4A). Furthermore, we measured the wettability of 5-HT and melanin blended scaffolding surface, which is an important parameter to determine their suitability as a biological support, was determined using a tensiometer by the quantitative mca-surement of contact angle (FIG. 4B-Bi). Surfaces displaying angles lower than 90° are considered hydrophilic (Agrawal et al., 2021; W et al., 2020). FIGS. 4B and 4Bi suggested that the observed mean±s.c.m. values of the contact angles for 5-HT-melanin and PHB composite scaffold (61.8±3.4°) and melanin blended PHB scaffold (60.6±2.5°) are higher than that of traditional fused silica glass surface (˜53°) (Ito et al., 2018) and PVA scaffolds (24.3±2.03°), but lesser than the PHB scaffold (100.03±1.2°). Nevertheless, the values are still <90° suggesting the hydrophilic nature of conductive 5-HT and melanin blended PHB scaffold.

### 2.4. Swelling and Degradation of Fibers

We measured the degree of swelling (swelling capacity (%)) of PHB and 5-HT-melanin-PHB scaffolds over a period of 12h (FIG. 8A-B). We found the (%) swelling capacity of 5-HT-mclanin-PHB fibers (497.81±10.94) to be significantly higher (** p<=0.01) than the one of PHB fibers (259.75±11.74) after 12h incubation in phosphate-buffered saline (PBS) (FIG. 8B). We also analyzed the degradation of PHB and 5-HT-melanin-PHB fibers by measuring weight loss after immersion in PBS for 10 days (FIGS. 8C and D). Preliminary degradation data plot for 10 days (FIG. 8E) suggested that blending of 5-HT and melanin increased (**** p<=0.0001) the biodegradation of scaffold (FIG. 8D) compared to PHB scaffold (FIG. 8C).

### 2.5. Biocompatibility

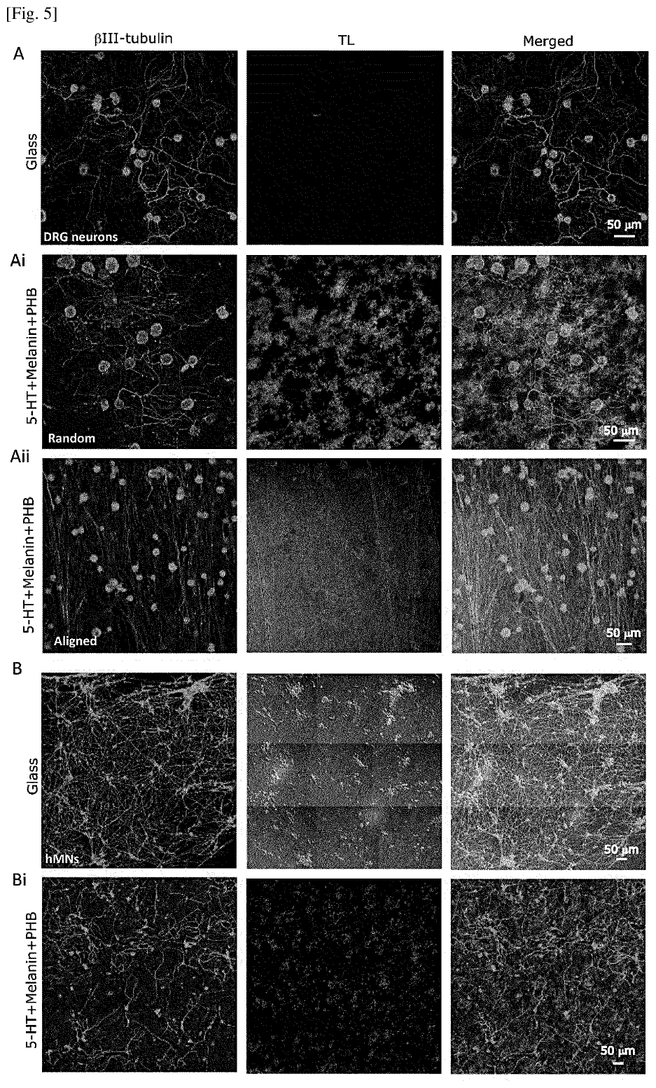

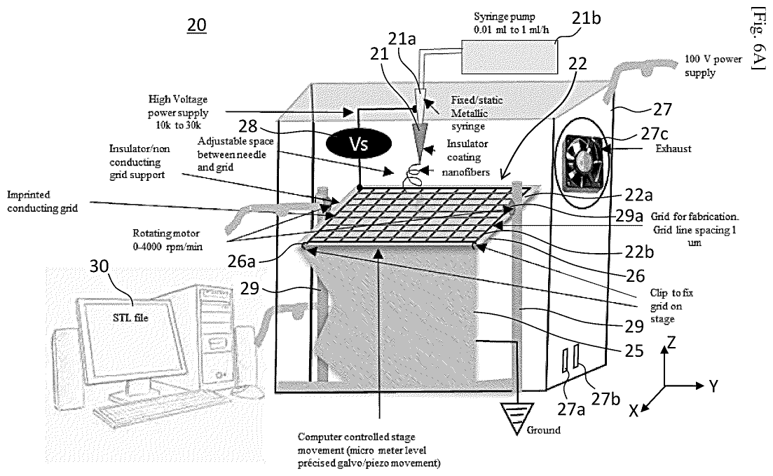

Because of the relevance of DRG and hMNs neurons in peripheral injury, spinal cord injuries and neuronal degeneration, we tested their biocompatibility with 5-HT-melanin and PHB composite scaffold (FIG. 5). Adult DRG neurons were dissected from 2-month-old mice, while hMNs were derived from human inducible pluripotent stem cells (IPS cells) and cultured on the scaffolds. For culture, 1 cm*1 cm*30 μm (L×W×H) scaffolds and control glass surface were coated with poly-L-Lysine and laminin, two ECM proteins, which are important in the successful attachment and growth of these neuronal types in culture. Subsequently, DRG neurons were seeded on the scaffolds (random and aligned; and glass surface (FIG. 5A), and allowed to grow for 48h and hMNs were allowed to grow for 7-days in vitro (DIV) (FIG. 5B). We then fixed the cells and stained with β-III tubulin antibody and analyzed with confocal microscope. We found that DRGs neurons could grow on our scaffolds and extend their neurites arbitrary when cultured on glass surface and random fibrous scaffolds (FIG. 5A and Ai); whereas neurons cultured on aligned fibers shows vectorized growth along the fibers (FIG. 5Aii). Similarly, hMNs could grow on our scaffolds comparably to traditional substrates like fused silica glass (FIG. 5B and Bi). No noticeable effects on neuronal viability or sign of axonal stress were observed when comparing nanofibers to the flat control surface. Thus, our data support the biocompatibility of 5-HT and melanin blended PHB scaffold and its suitability for mammalian cell cultures and to use as an implant.

### 3. Design of Advanced Fabrication Setup:

We are developing electrically conducting, biocompatible and biodegradable implants for the purpose of tissue engineering to address the long-lasting medical problems such as SCI, nerve injuries (NI) and neo-tissue formation for the treatment of the deep wound (>=5 mm) usually formed after the accidents or during critical surgeries such as cortical brain surgery and open-heart surgery. Researchers found that the combined use of biomaterials with and without the use of stem cells have shown the potential to replace the conventional inefficient methods to deal said medical problems. Recent advancements in the fabrication technology enabled us to fabricate 2D scaffolds and circuitries with a high precision range from few hundreds of nanometers such as 2-photon polymerization (2-PP) laser lithography system. However, when it comes to the cost-effective fabrication of 3D structures from nanometer to few millimeters dimension with user defined shape/size and specific texture and with the high level of precision, over a short-time period; developing such a tool or state of the art technology remains a big challenge to the scientific community. On one hand, conventional electrospinning setup allows faster fabrication but is mainly limited to development of 2D scaffolds (ranging from mm to cm with limited thickness) and doesn't allow any control over scaffolding parameters such as fiber diameter and inter-fiber space. On the other hand, 3D-lithography allows precise control over parameter (˜200 nm) but the size of scaffold is restricted (only few millimeters) (Agrawal et al., 2021). Further, it is a laborious and expensive method, and requires relatively longer print time. Considering all the shortcomings of existing fabrication techniques, we decided to develop a hybrid nanofabrication system amal-gamating the principles of electrospinning(ES) and 3D-bioprinting based on a conducting grid pattern to overcome the said limitations. FIG. 6A-D represent the proposed design of the hybrid fabrication system. FIG. 6A shows the detailed grid-based technology of the proposed fabrication setup. Varying the patterns of grid geometry and type of collector and grid spacing parameters attached with a piezo-enabled (X, Y and Z) stage (FIG. 6B-D), enables the fabrication of a user defined computer-aided-design in ES-mode, which is not possible with the existing ES-setups. In addition, 3D-bioprinting technique can be easily integrated in this system by controlling the size of the needle/nozzle, flow rate of polymeric solution and flow of the CO₂/air passing through the polymeric solution. This would make this setup a cost-effective 3D fabrication technique. Our setup would also allow for the fabrication of 3D-hydrogels for therapeutic purposes. Further, integration of multi-nozzle will allow us to use semisolid viscous solutions to fabricate complex 3D structure with high level of precision, giving us an upper hand over existing fabrication technique.

### DISCUSSION

Every year several million people worldwide suffer from SCI (Kang et al., 2017), 15-40% of the cases also involving PNI (S. Chen et al., 2015). Over the last decade, regenerative medicine has achieved rapid and promising advancements in neural tissue engineering and stem cell therapy (McMurtrey, 2015; Rajabzadeh et al., 2019). Effective therapeutic strategies, for the treatment of SCI and PNI, are still limited (Cristante et al., 2012; Hussain et al., 2020), mainly including the use of implants to stabilize the spinal cord, auto grafting, and systemic injection of growth factors (Jendelova, 2018). Motor/sensory neuron loss of function, neuronal degeneration and mismatch of damaged nerve and graft dimensions are known drawbacks of these ap-proaches. Meta-analysis of more than 70 preclinical studies suggests that combination of cell therapy with various scaffolds improves function restoration when compared to cell therapy alone (Baklaushev et al., 2019). Nonetheless, it is still a major challenge to engineer an ideal nanofibrous scaffold that provides an attractive clinical alternative to nerve auto grafts and semiconductor implants for SCI (Baklaushev et al., 2019; Boni et al., 2018). We fabricated 5-HT-melanin blended PHB composite conductive scaffolds for neural tissue engineering. SEM analysis confirmed successful fabrication of ˜290 nm (FIG. 1f) diameter 5-HT and melanin blended PHB fibers. Recently, increasing efforts have been directed to the development of biodegradable and conductive scaffold (Agrawal et al., 2022). Addition of melanin and 5-HT has not only increased the biodegradation capabilities of nanofibers but also made our fibers conducting in nature (FIG. 4A). Conductive nature of the scaffolds in physiological conditions will provide electrical cues for the accelerated and aligned growth of neurites. Further, combinatorial effect of the fiber rough surface, conductive nature and Laminin coating facilitated the attachment and survival of mouse sensory neuron (Agrawal et al., 2022) and human motor neurons (FIG. 5).

We also fabricated aligned fibers for creating a mechanical constraint to promote the vectorized growth of axon (FIG. 1B-1E; middle panel) (Agrawal et al., 2021). Indeed, aligned fibers can promote the vectorized growth (FIG. 5Aii) of residual axons at the site of injury and help repair the function of the damaged nerve tissue (Hadlock et al., 2000). Therefore, fabrication of aligned fibers was done in preparation of the engineering of implants for use in vivo, where directing axonal growth along a main axis would be advantageous. Taken together our study demonstrate the advantages of 5-HT, melanin and PHB aligned/random scaffold/conduits/implants to promote repair damaged nerve tissue at the site of injury as a therapeutic approach to increase tissue regeneration. Lastly, we propose a design for a hybrid grid based fabrication setup (FIG. 6) allowing for 3D scaffold fabrication overcoming the limitations of existing fabrication system. We believe that this setup will be a cost effective way to fabricate aligned and 3D scaffolds for patient specific fabrication of implants for medical use, which will be beneficial for clinical applications.

1-12. (canceled)

13. A system for fabricating a fibrous assembly comprising:
a nozzle configured to be supplied with a spinning liquid; and
a collector arranged at a distance from the nozzle, wherein
the collector includes a target electrode with a pattern and when a voltage is applied between the nozzle and the target electrode, the spinning liquid is discharged in an electrically charged state toward the target electrode to form fibers, which are deposited as a fibrous assembly on the collector, such that an alignment, diameter, orientation and inter-fiber space of the fibers in the fibrous assembly can be controlled by varying the pattern of the target electrode.

14. The system according to claim 13, further comprising a moving mechanism configured to move one or both of the nozzle and the collector relative to each other, such that a shape and size of the fibrous assembly can be controlled by moving one or both of the nozzle and the collector relative to each other during discharge of the spinning liquid.

15. The system according to claim 14, wherein
the moving mechanism is configured to move one or both of the nozzle and the collector relative to each other in an X direction and a Y direction, both of which are perpendicular to a discharge direction of the nozzle.

16. The system according to claim 14, wherein
the moving mechanism is configured to move one or both of the nozzle and the collector relative to each other in a Z direction parallel to a discharge direction of the nozzle.

17. The system according to claim 14, wherein
the moving mechanism includes a motor, such that the amount of movement of one or both of the nozzle and the collector can be controlled with a precision of 200 nm or less.

18. The system according to claim 14, further comprising a control unit configured to control an operation of the moving mechanism, wherein
the control unit includes:
a data reception unit configured to receive CAD data of a fibrous assembly to be manufactured,
a calculation unit configured to calculate timing and amount of movement of one or both of the nozzle and the collector according to the CAD data, and
a signal transmitting unit configured to transmit a control signal according to the calculated timing and amount of movement to the moving mechanism.

19. The system according to claim 13, comprising a plurality of the nozzles.

20. The system according to claim 19, wherein
the plurality of nozzles each have a different nozzle diameter, such that the fibrous assembly can contain a plurality of types of fibers with different diameters.

21. The system according to claim 19, wherein
the plurality of nozzles each are supplied with a different spinning liquid or a combination of a spinning liquid, a gas and a solvent, such that the fibrous assembly can contain a plurality of types of fibers with different properties by simultaneously discharging the spinning liquids or combinations from the plurality of nozzles.

22. The system according to claim 13, further comprising: a spinning chamber, the nozzle and the collector being located therein; and at least one of a humidity controller and a temperature sensor inside the spinning chamber.

23. The system according to claim 13, wherein the collector has a grid pattern, a concentric rectangular pattern, a concentric circular pattern, a solid planar shape, or a cylindrical shape.

24. The system according to claim 13, wherein the collector is an exchangeable or removable collector.

25. A collector with a target electrode for use in the system according to claim 1.

26. A method for fabricating a fibrous assembly comprising:
determining a pattern of a target electrode on a collector based on a desired alignment, diameter, orientation and inter-fiber space of fibers in a fibrous assembly;
arranging the collector conducting in nature and having the target electrode at a distance from a nozzle configured to be supplied with a spinning liquid; and
applying a voltage between the nozzle and the target electrode, so as for the spinning liquid to be discharged in an electrically charged state toward the target electrode to form fibers, which are deposited as the fibrous assembly on the collector.

27. A method of producing a fiber, comprising:
arranging a collector having a target electrode at a distance from a nozzle configured to be supplied with a spinning liquid; and
applying a voltage between the nozzle and the target electrode, so as for the spinning liquid to be discharged in an electrically charged state toward the target electrode to form fibers, wherein the spinning liquid comprises a biocompatible polymer.

28. (canceled)

29. The method according to claim 27, wherein the biocompatible polymer is a biodegradable biocompatible polymer.

30. The method according to claim 27, wherein the biocompatible polymer is natural biodegradable biocompatible polymer.

31. The method according to claim 27, wherein the biocompatible polymer is selected from the group consisting of PHA, PHB, or poly (3-hydroxy butyrate)), chitosan, pectin, gelatin, agar-agar, polycaprolactone and collagen.

32. The method according to claim 27, wherein the biocompatible polymer is loaded with one or more of drugs, neuro hormones, neuro peptides, serotonin (5-HT), other hormones, other peptides, or melanin.
